(19) United States
(12) Patent Application Publication
(10) Pub. No.: US 2017/0281891 A1
(43) Pub. Date: Oct. 5, 2017

(54) TRACHEA MONITORING TUBE AND ENDOTRACHEAL CATHETER INCORPORATING THE SAME

(71) Applicant: CHIMEI MEDICAL CENTER, Tainan City (TW)

(72) Inventors: JEN-YIN CHEN, TAINAN CITY (TW); KUO-MAO LAN, TAINAN CITY (TW)

(21) Appl. No.: 15/084,928

(22) Filed: Mar. 30, 2016

(51) Int. Cl. A61M 16/04

(52) U.S. Cl. CPC A61M 16/0488

(57) ABSTRACT

A trachea monitoring tube and an endotracheal catheter incorporating the same are characterized in that the trachea monitoring tube is located outside a breathing tube of the endotracheal catheter. More specifically, the breathing tube has a first end portion and a second end portion, the latter of which is inserted into a patient's respiratory duct during use; and the trachea monitoring tube has a monitoring end fixed to the breathing tube and spaced from the first end portion of the breathing tube by a predetermined distance. An endoscope can be inserted into the trachea monitoring tube in order to observe from outside the breathing tube how the breathing tube is inserted into the patient's respiratory duct.

### BACKGROUND OF THE INVENTION

### 1. Technical Field

The present invention relates to a trachea monitoring tube and an endotracheal catheter incorporating the same. More particularly, the present invention relates to a structure by which the insertion of a breathing tube into a patient's respiratory duct can be observed from outside the breathing tube.

### 2. Description of Related Art

Tracheal intubation is an effective medical procedure for sustaining the respiratory function of one who has stopped breathing, and it is important to ensure that the breathing tube stays at the correct position once inserted into the respiratory duct.

Taiwan Patent Application Publication No. 201416049, entitled “UNIVERSAL ENDOTRACHEAL TUBE ADAPTOR OF ENDOSCOPY SYSTEM”, discloses a breathing tube incorporating an endoscope. As the endoscope is directly inserted into the breathing tube to enable observation of the movement of the breathing tube along the tube's moving path, the observation is not as accurate as if the moving path is observed from outside the breathing tube. Moreover, in cases where another instrument, such as a suction catheter, is required to be inserted into the breathing tube, there may be insufficient space through which the endoscope can pass.

Taiwan Patent No. M429482, entitled “TRACHEAL INTUBATION DEVICE”; U.S. Pat. No. 4,306,547, entitled “RIGID FIBEROPTIC LARYNGOSCOPE”; and US Patent Application Publication No. 20070106121, entitled “INTUBATION ASSISTANCE APPARATUS AND INTUBATION ASSISTANCE USED IN THE APPARATUS”, disclose supporting an endoscope and a breathing tube by means of a supporting base so that the moving path of the breathing tube can be observed from outside the tube via the endoscope. These prior art devices, however, are structurally complicated. Not only must the supporting base have a passageway for the breathing tube as well as the endoscope, but also the breathing tube has to be inserted further into the respiratory duct with respect to the supporting base after the supporting base is in place. Besides, these prior art breathing tubes do not have a marked portion with which a surgeon can observe the inserted length of a breathing tube into the respiratory duct. The prior art, therefore, leaves room for improvement.

### BRIEF SUMMARY OF THE INVENTION

To overcome the aforementioned drawbacks of the prior art, the present invention provides an endotracheal catheter incorporating a trachea monitoring tube.

The endotracheal catheter includes a breathing tube and a trachea monitoring tube. The breathing tube has a first end portion and a second end portion. The breathing tube also has a marked portion adjacent to the first end portion. The trachea monitoring tube serves only to provide an endoscope passageway through which an endoscope can be inserted. The trachea monitoring tube has a monitoring end fixed to an exterior portion of the breathing tube and spaced from the first end portion of the breathing tube by a predetermined distance. As the breathing tube enters the respiratory duct, the trachea monitoring tube moves simultaneously with the breathing tube.

Furthermore, the monitoring end of the trachea monitoring tube is 0.5 cm to 3 cm, or preferably 1 cm, away from the marked portion.

Furthermore, the breathing tube has an upper portion and a lower portion, and the monitoring end of the trachea monitoring tube is located at the lower portion of the breathing tube. In particular, the monitoring end of the trachea monitoring tube is at a 5 o'clock or 7 o'clock position.

Furthermore, the trachea monitoring tube has a rear end and extends along and around the breathing tube in a spiral manner such that the rear end is located at a 9 o'clock or 3 o'clock position of the second end portion of the breathing tube.

The present invention also provides a trachea monitoring tube configured to be fixed to a breathing tube, wherein the breathing tube has a first end portion and a second end portion. The trachea monitoring tube has a monitoring end and a fixing element. The fixing element is configured to fix the monitoring end to the breathing tube such that the monitoring end is spaced from the first end portion of the breathing tube by a predetermined distance.

Furthermore, the fixing element is one of a C-clamp, an attaching device, and a shrink wrap.

The foregoing technical features can produce the following effects:

1. By placing an endoscope into the trachea monitoring tube, a surgeon can observe how the breathing tube enters a patient's respiratory duct from outside the breathing tube.

2. As the monitoring end of the trachea monitoring tube is fixed to the breathing tube and moves with the breathing tube at the same time, and thanks to the marked portion of the breathing tube, a surgeon can easily know the inserted length of the breathing tube into a patient's respiratory duct.

3. The present invention features structural simplicity because the trachea monitoring tube only has to provide the endoscope passageway and can be fixed to a common breathing tube by nothing more than the fixing element. Therefore, the present invention can be implemented at low cost and has great potential of being widely used.

### BRIEF DESCRIPTION OF THE SEVERAL VIEWS OF THE DRAWINGS

FIG. 1 is a perspective view of a trachea monitoring tube according to an embodiment of the present invention;

FIG. 2 is a perspective view of an endotracheal catheter incorporating a trachea monitoring tube according to an embodiment of the present invention;

FIG. 3 schematically shows how the present invention makes it possible to observe from outside a breathing tube how the breathing tube is inserted into the respiratory duct;

FIG. 4 is another schematic drawing showing how the present invention makes it possible to observe from outside a breathing tube how the breathing tube is inserted into the respiratory duct; and

FIG. 5 is still another schematic drawing showing how the present invention makes it possible to observe from outside a breathing tube how the breathing tube is inserted into the respiratory duct.

### DETAILED DESCRIPTION OF THE INVENTION

The present invention integrates the aforesaid technical features into a trachea monitoring tube and an endotracheal catheter incorporating the same, whose major effects are demonstrated below with reference to an illustrative embodiment.

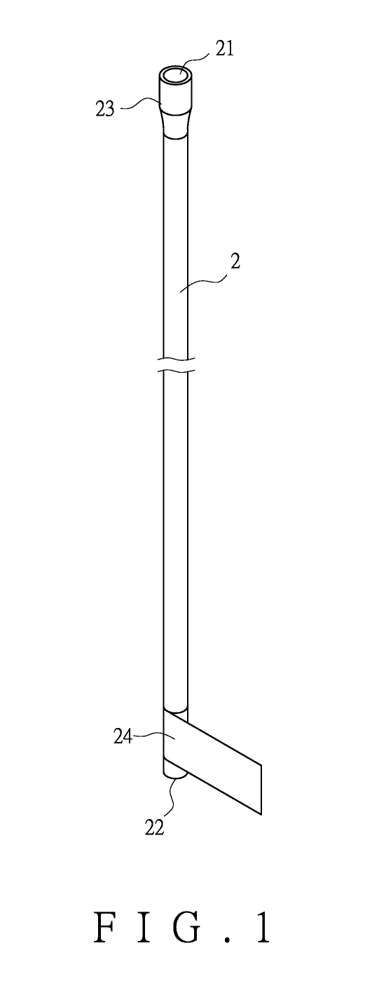

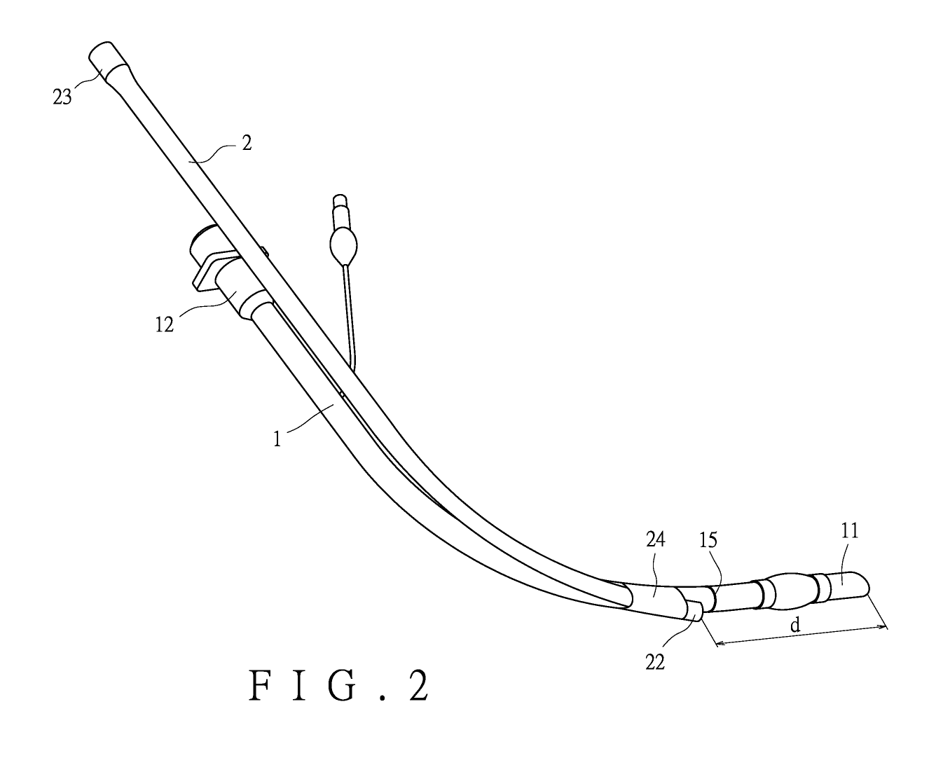

As shown in FIG. 1 and FIG. 2, this embodiment includes a breathing tube 1 and a trachea monitoring tube 2.

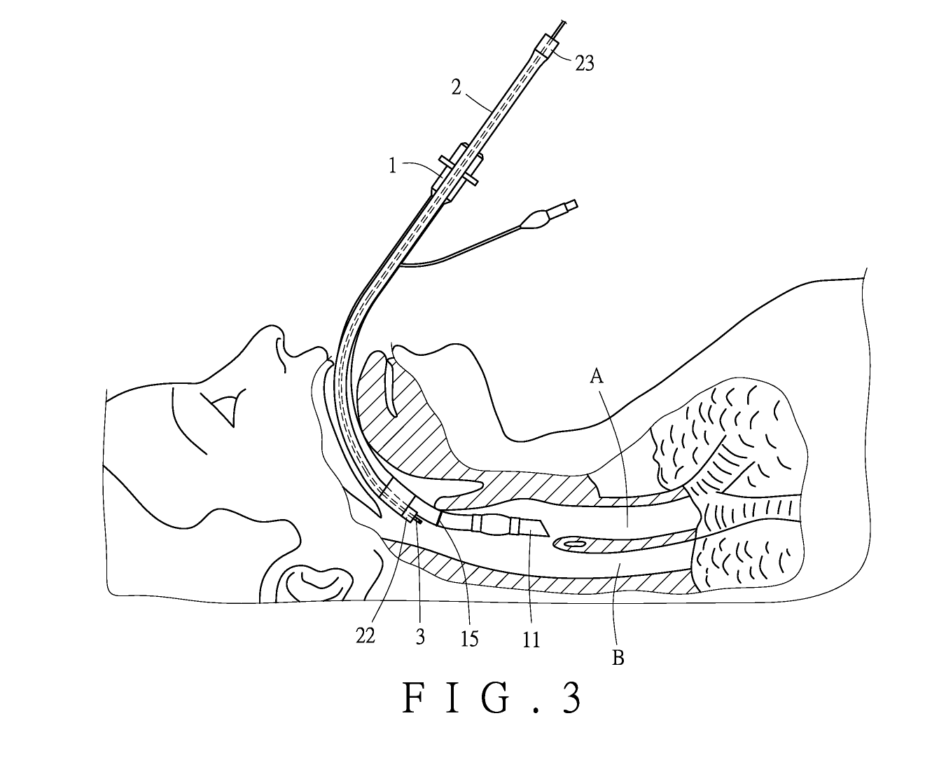

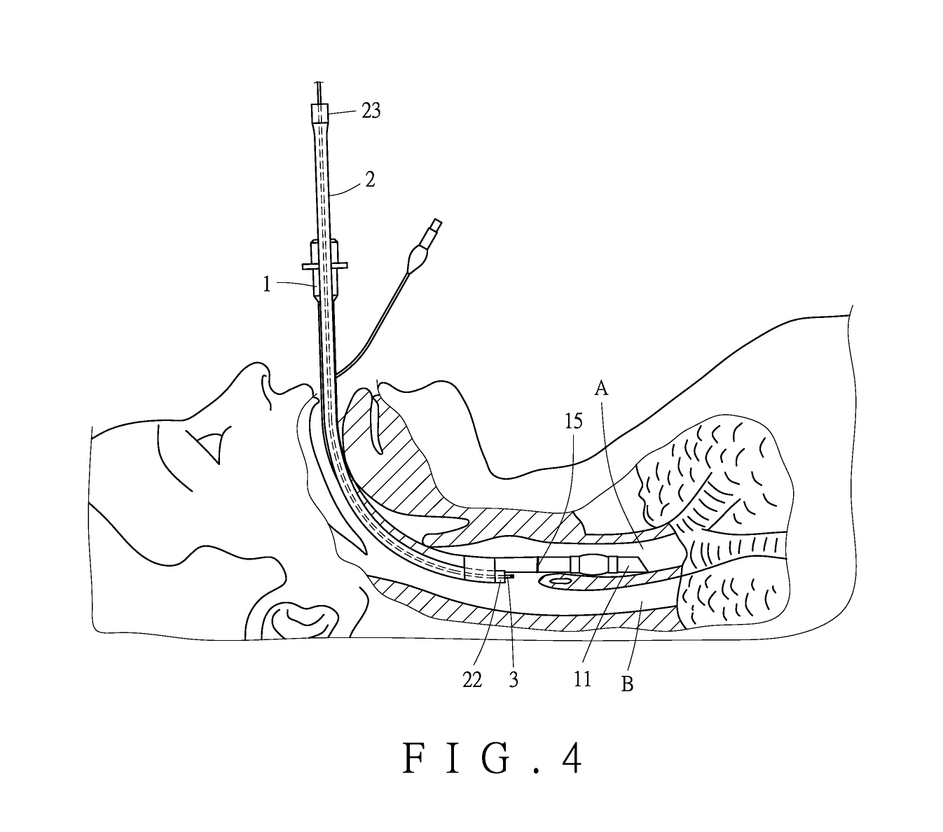

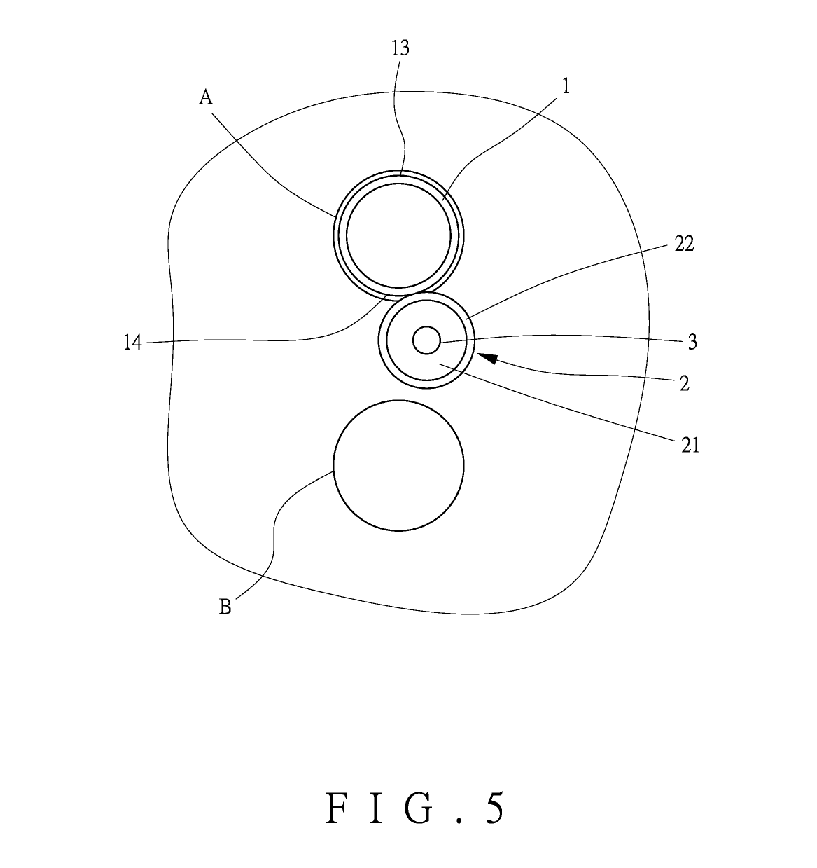

The breathing tube 1 has a first end portion 11, a second end portion 12 opposite the first end portion 11, an upper portion 13, and a lower portion 14 opposite the upper portion 13 (see FIG. 5 for the upper portion 13 and the lower portion 14). The breathing tube 1 also has a marked portion 15 adjacent to the first end portion 11. The trachea monitoring tube 2 serves only to provide an endoscope passageway 21 through which an endoscope 3 can be inserted (see FIG. 3, FIG. 4, or FIG. 5 for the endoscope 3).

The trachea monitoring tube 2 has a monitoring end 22 and a rear end 23 opposite the monitoring end 22. The trachea monitoring tube 2 has a fixing element 24 for fixing the monitoring end 22 to an exterior portion of the breathing tube 1. For example, the fixing element 24 can be a C-clamp, an attaching device, or a shrink wrap. As the human respiratory duct A is located in front of the esophagus B, the monitoring end 22 is fixed to the lower portion 14 of the breathing tube 1 to facilitate observation during tube insertion. Preferably, the monitoring end 22 is at a 5 o'clock or 7 o'clock position and is spaced from the first end portion 11 of the breathing tube 1 by a predetermined distance d. Moreover, the monitoring end 22 is spaced from the marked portion 15 by a distance ranging from 0.5 cm to 3 cm, preferably 1 cm. The trachea monitoring tube 2 extends along and around the breathing tube 1 in a spiral manner such that the rear end 23 is located at a 9 o'clock or 3 o'clock position of the second end portion 12 of the breathing tube 1 to facilitate the closing of jaws.

Referring to FIG. 3 and FIG. 4, a surgeon can place the first end portion 11 of the breathing tube 1 into the respiratory duct A (which is in front of the esophagus B) of a patient who needs tracheal intubation, and then insert the endoscope 3 through the rear end 23 of the trachea monitoring tube 2 into the endoscope passageway 21 until the monitoring end 22 of the trachea monitoring tube 2 is reached. Thus, not only can the patient's respiratory duct A and esophagus B be clearly observed, but also the moving direction of the breathing tube 1 can be observed from outside the breathing tube 1 in order to place the breathing tube 1 precisely in the respiratory duct A. By observing the marked portion 15 of the breathing tube 1, the surgeon can make sure that the breathing tube 1 has reached the desired depth. If the breathing tube 1 is inserted too deep, the respiratory duct A may get hurt as a result, but the breathing tube 1 may fall out if not inserted deep enough. Since the trachea monitoring tube 2 advances along with the breathing tube 1, the predetermined distance d therebetween remains constant, which facilitates the surgeon's observation.

Referring to FIG. 5, the monitoring end 22 of the trachea monitoring tube 2 is fixed at a 5 o'clock or 7 o'clock position of the breathing tube 1 so that observation can be made with ease during tube insertion.

According to the present invention, the trachea monitoring tube 2 only has to provide the endoscope passageway 21. Further, the trachea monitoring tube 2 only has to incorporate the fixing element 24 in order to be fixed to a common breathing tube. This allows the present invention to be implemented at low cost and therefore have great potential of being widely used.

The foregoing description should be able to enable a person of ordinary skill in the art to fully understand the operation, use, and effects of the present invention. The embodiment described above, however, is only one preferred embodiment of the invention and is not intended to be restrictive of the scope of the invention. All simple equivalent variations and modifications made according to the appended claims and the disclosure of the present specification should fall within the scope of the present invention.

What is claimed is:

1. An endotracheal catheter incorporating a trachea monitoring tube, for use in combination with an endoscope during tracheal intubation, the endotracheal catheter comprising:
a breathing tube having a first end portion and a second end portion, the breathing tube also having a marked portion adjacent to the first end portion; and
a trachea monitoring tube providing an endoscope passageway through which the endoscope can extend, the trachea monitoring tube having a monitoring end, the monitoring end being fixed to an exterior portion of the breathing tube, the monitoring end and the first end portion of the breathing tube being spaced by a predetermined distance, wherein when the breathing tube enters a respiratory duct, the trachea monitoring tube is moved synchronously with the breathing tube.

2. The endotracheal catheter of claim 1, wherein the monitoring end of the trachea monitoring tube is spaced from the marked portion by a distance ranging from 0.5 cm to 3 cm.

3. The endotracheal catheter of claim 2, wherein the monitoring end of the trachea monitoring tube is spaced from the marked portion by 1 cm.

4. The endotracheal catheter of claim 1, wherein the breathing tube has an upper portion and a lower portion, and the monitoring end of the trachea monitoring tube is located at the lower portion of the breathing tube.

5. The endotracheal catheter of claim 4, wherein the monitoring end of the trachea monitoring tube is located at a 5 o'clock or 7 o'clock position.

6. The endotracheal catheter of claim 5, wherein the trachea monitoring tube has a rear end, and the trachea monitoring tube extends along and around the breathing tube in a spiral manner such that the rear end is located at a 9 o'clock or 3 o'clock position of the second end portion of the breathing tube.

7. A trachea monitoring tube configured to be fixed to a breathing tube, wherein the breathing tube has a first end portion and a second end portion, the trachea monitoring tube comprising a monitoring end and a fixing element for fixing the monitoring end to the breathing tube such that the monitoring end and the first end portion of the breathing tube are spaced by a predetermined distance.

8. The trachea monitoring tube of claim 7, wherein the fixing element is a C-clamp, an attaching device, or a shrink wrap.
